(19) United States
(12) Patent Application Publication
(10) Pub. No.: US 2004/0003648 A1
(43) Pub. Date: Jan. 8, 2004

(54) Determination of carbon and silicon contents in molten iron

(76) Inventors: Mayuki Morinaka, Shizuoka (JP); Tsuyoshi Okuzono, Kogoshima (JP)

Correspondence Address: MUSERLIAN AND LUCAS AND MERCANTI, LLP, 475 PARK AVENUE SOUTH, NEW YORK, NY, 10016, US

(21) Appl. No.: 09/497,598

(22) Filed: Feb. 3, 2000

(30) Foreign Application Priority Data
Feb. 4, 1999 (JP) 11-63.746

(51) Int. Cl. G01N 25/04; G01N 33/20

(52) U.S. Cl. 73/19.07; 374/45; 374/26

(57) ABSTRACT

A nethod for determining carbon and silicon contents in a molten cast iron is disclosed. The method consists of steps for carrying out thermal analysis the molten cast iron poured into a first sampling vessel to which a small amount of tellurium is added and a second sampling vessel to which a small amount of silicon is added, and comparing the primsary crystal temperatures in the molten cast iron in the first and second sampling vessels.

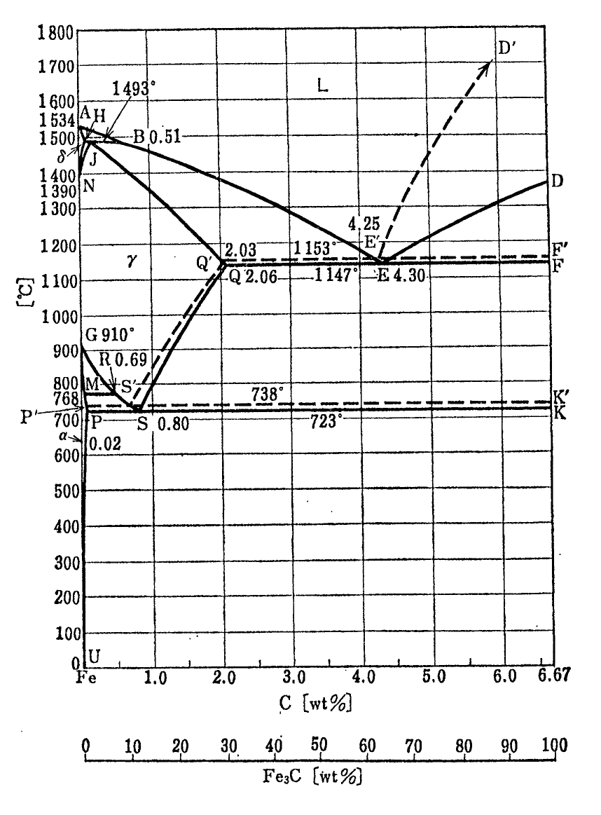

### BACKGROUND OF THE INVENTION

This invention relates to a method of determination of carbon and silicon contents in molten iron, and more particularly to a method of predicting carbon and silicon contents in molten cast or pig iron by thermal analysis. Hereinafter, if there is no need to define cast iron or pig iron, it is referred to as “cast iron”.

Molten iron prepared by a cupola or molten pig iron melted in a blast furnace is referred to as “eutectic structure”, and the carbon contents in the eutectic structure of molten metal are converted into hypoeutectic or hypereutectic iron depending on the condition of the operation with respect to the amounts of foundry coke and the wind to be blown in the furnace.

In some cases, additives such as silicon may be added to molten cast iron in order to obtain a desired composition, and in the case of pig iron, molten pig iron is transferred to a converter to remove carbon and silicon so as to convert it to steel. In order to obtain steel from molten pig iron, the quality of air to be blown into the converter should be controlled according to the carbon and silicon contents.

Usually, an emission spectroscopic analysis is applied to measure the carbon or silicon contents in molten cast or pig iron.

In the emission spectroscopic analysis portion of molten metal is ladled into a sampling vessel, and the sample is solidified. The surface of solidified sample is polished with extremely fine abrasive, and then the polished surface is analyzed.

In the past, the thermal analysis is applied to measure the carbon and silicon contents in molten cast iron. In spite of the carbon equivalent value (CE) of each of hypoeutectic and hypereutectic compositions in molten cast iron is different each other, in a cooling curve obtained by the analysis, the primary crystal temperature of both compositions are often plotted on the same point. Consequently, the thermal analysis for measuring the carbon and silicon contents in molten cast iron are usually restricted to a few hypoeutectic or hypereutectic cast iron.

In an exact hypereutectic cast iron, either graphite or cementite (Fe₃C) is crystallized from the molten cast iron. However, the primary crystal temperatures of graphite and cementite are different each other as clear from the phase diagram, and also the carbon contents in graphite and cementite are different.

The primary crystal of hypoeutectic is only austenite cast iron, and the carbon content may be measured by the hypoeutectic temperature of austenite.

As clear in the phase diagram of Fe—C molten cast iron, in the hypereutectic region, there are two liquidus lines (AB and BE). The carbon content in the molten iron may be measured by the primary crystal temperature, but as there is not enough of crystal nucleus, the liquidus line of cementite will not be appeared on the diagram. Therefore, an actual primary temperature appears on between the two liquidus lines.

If molten cast iron is hypoeutectic cast iron, either graphite or cementite is crystallized. If graphite nuclear contains rather excess in the molten cast iron, the graphite liquidus line will be appeared in the phase diagram of Fe—C molten cast iron. On the other hand, if there is not enough of graphite nuclear in molten cast iron, it cools in supercooling condition, and the primary crystal temperature appears on lower than the liquidus line.

In the absent of graphite nuclear in molten cast iron, cementite is crystallized. However, nuclear material in actual molten cast iron will not be appeared on the phase diagram as a liquidus line of cementite.

Accordingly in order to measure the carbon content in actual molten cast iron, it is necessary to add a good enough of graphite nuclear material in it, or to disappear graphite nuclear material which exists therein.

In the light of the foregoing, it is impossible to measure or determine the carbon or silicon content in molten cast or pig iron by the usual method of thermal analysis, if the molten metal is hypereutectic state.

It is a principal object of the invention to provide a method of determination of carbon and silicon contents in molten cast iron or pig iron.

Other objects of the present invention will appear hereinafter.

### BRIEF DESCRIPTION OF THE DRAWING

The FIGURE is a phase diagram of Fe—C molten cast iron.

### DETAILED DESCRIPTION OF THE INVENTION

For the attainment of these objects, a method for determining carbon and silicon contents in molten cast iron or pig iron according to the present invention includes: steps for

(1) preparing a first sampling vessel for use in thermal analysis of molten metal in which a small amount of tellurium (Te) is placed;

(2) preparing a second sampling vessel in which a small amount of silicon (Si) is placed;

(3) ladeling molten metal into the first and second sampling vessels simultaneously;

(4) conducting thermal analysis of the molten cast iron in the first sampling vessel;

(5) conducting thermal analyze of the molten cast iron in the second sampling vessel;

(6) comparing a primary crystal temperature of the molten metal in the first sampling vessel with one of the molten metal in the second sampling vessel.

According to the present invention, molten cast iron is thermally analyzed by using the first sampling vessel in which a small amount of tellurium is added and the second sampling vessel in which a small amount of silicon is added, and then the primary crystal temperatures of the molten cast iron in both vessels are compared with each other.

As the result of the above comparison, if the primary crystal temperature of the molten cast iron in the first vessel is higher than that of the molten cast iron in the second vessel, it may be decided that the molten cast iron is hypoeutectic cast iron. Contrary to this, if the primary crystal temperature of the molten cast iron in the second vessel is higher than one of the first vessel, it is decided that the molten cast iron is hypereutectic.

Further, as in the first sampling vessel contains a small amount of tellurium, the primary crystals in the molten cast iron are formed to cementite. Consequently, when the sample is decided to hypoeutectic cast iron, the carbon content in the sample will be easily known by its calibration carve.

On the other hand, when the sample of molten cast iron is ladled into the second vessel containing silicon, the carbon equivalent of the molten cast iron will be increased by the action of silicon. Accordingly, as shown in the phase diagram the primary crystal temperature of the molten cast iron is approached to eutectic temperature which is lower than the primary crystal temperature of the molten cast iron in the first vessel.

When the given sample is hypereutectic cast iron, the primary crystal temperature of the sample in the second vessel is higher than one of the first sampling vessel.

After the determination of either hypereutectic or hypoeutectic cast iron, the carbon content of the molten cast iron in the first vessel may be measured by the calibration curve.

Further, for the reason that the first sampling vessel contains tellurium, even if molten cast iron in the first vessel hypereutectic cast iron, it is solidified in white pig iron, and may obtain a fixed eutectic temperature for analyzing carbon and silicon.

Experiment

Samples of molten cast iron No. 1 to No. 5 which are chemically analyzed with respect to the carbon (C) content and silicon (Si) content, and the carbon equivalent value (CE) as shown in TABLE I were respectively maintained at the temperature of 1500° C.

These samples were ladled into the first sampling vessel to which a small amount of tellurium was added, the second sampling vessel to which a small amount of silicon such as ferrosilicons (for example Fe-75% Si) or Fe-40% Si-10% Ca was added,

and the third sampling vessel which is free of additive, and then the samples were thermally analyzed for obtaining cooling curves to measure the primary crystal and eutectic temperature, carbon and silicon contents, and carbon equivalent, respectively.

TABLE I

| Sample | No. 1 | No. 2 | No. 3 | No. 4 | No. 5 |
|---|---|---|---|---|---|
| C (%) | 4.75 | 4.4 | 4.1 | 3.75 | 3.5 |
| Si (%) | 1.4 | 1.4 | 1.4 | 1.4 | 1.4 |
| CE | 5.2 | 4.9 | 4.6 | 4.3 | 4.0 |

The results of thermal analysis of the samples No. 1 to No. 5 are shown in TABLE II, and TABLE III shows the carbon and silicon contents in these samples.

TABLE II
(Results of Thermal Analisis)

| | | | | | |
|---|---|---|---|---|---|
| CE | 4.0 | 4.3 | 4.6 | 4.9 | 5.2 |

Primary Crystal Temp.

| | | | | | |
|---|---|---|---|---|---|
| 1st Vessel | 1192 | 1150 | 1124 | 1164 | 1212 |
| 2nd Vessel | 1189 | 1152 | 1167 | 1223 | 1258 |
| 3rd Vessel | 1195 | 1157 | 1160 | 1185 | 1241 |

Eutectic Temperature

| | | | | | |
|---|---|---|---|---|---|
| 1st Vessel | 1123 | 1123 | 1123 | 1123 | 1122 |
| 2nd Vessel | 1150 | 1152 | 1153 | 1154 | 1153 |
| 3rd Vessel | 1129 | 1133 | 1147 | 1149 | 1151 |

TABLE III

| Sample | No. 1 | No. 2 | No. 3 | No.4 | No.5 |
|---|---|---|---|---|---|
| C (%) | 4.7 | 4.4 | 4.1 | 3.8 | 3.5 |
| Si (%) | 1.4 | 1.4 | 1.4 | 1.4 | 1.4 |
| CE | 5.2 | 4.9 | 4.6 | 4.3 | 4.0 |

In comparison of the TABLE I with TABLE III, the results according to the thermal analysis agree approximately with the results of the chemical analysis.

According to the present invention, the same sample of molten cast iron is analyzed thermally with using the first sampling vessel placed a small amount of tellurium and the second sampling vessel placed a small amount of silicon, and primary crystal temperatures of molten cast iron in the first and second sampling vessels are compared with each other.

What is claimed us:

1. A method for detemining carbon and silicon contents in molten cast iron consisting of steps for:
(1) preparing a first sampling vessel for use in samal analysis of the molten cast iron to which a small amount of tellurium is added;
(2) preparing a second sampling vessel in which a small ampount of silicon is added;
(3) ladling said molten cast iron into said first and secon vessels simulatenaously;
(4) conducting thermal analysis of said molten cast iron in said first vessel;
(5) conducting thermal analysis of said molten cast iron in said second vessel; and
(6) comparing a primary crystal temperature of said molten cast iron in said first vessel with one of said molten cast iron in said second vessel.

2. A method for determining carbon and silicon contents in molten cast iron claimed in claim 1 in which said silicon is selected from ferrosilicons.

3. A method for determining carbon and silicon contents in molten cast iron claimed in claim 1 in which said silicon is Fe-75% Si.

4. A method for determining carbon and silicon contents in molten cast iron claimed in claim 1 in which said silicon is Fe-40% Si-10% Ca.
